(19) United States
(12) Patent Application Publication
(10) Pub. No.: US 2011/0046173 A1
(43) Pub. Date: Feb. 24, 2011

(54) COMBINATION ANALGESIC OPIOID PAIN THERAPY

(76) Inventors: Warren Charles Stern, Plymouth, MA (US); Patricia Tewes Richards, West New York, NJ (US)

Correspondence Address: KING & SPALDING, 1180 PEACHTREE STREET , NE, ATLANTA, GA, 30309-3521, US

(21) Appl. No.: 12/567,209

(22) Filed: Sep. 25, 2009

Related U.S. Application Data
(60) Provisional application No. 61/236,255, filed on Aug. 24, 2009.

(51) Int. Cl. A61K 31/485

(52) U.S. Cl. 514/282

(57) ABSTRACT

Provided are pharmaceutical compositions and methods for the alleviation of pain in a patient with ratios of morphine and oxycodone that provide lower incidence of adverse side effects compared to equi-analgesic doses of morphine and oxycodone alone. The pharmaceutical compositions comprise an analgesic amount of morphine and an analgesic amount of oxycodone, or pharmaceutically acceptable salts thereof, in ratios of about 3 to 2 to about 1.25 to 1, morphine to oxycodone by weight.

### CROSS REFERENCE TO RELATED APPLICATIONS

This application claims the benefit of U.S. Provisional Application No. 61/236,255 filed Aug. 24, 2009, which is incorporated herein by reference in its entirety.

### FIELD OF THE INVENTION

This invention is directed to pharmaceutical compositions comprising optimal combinations of morphine and oxycodone that provide synergistic efficacy and lower incidence of undesired side effects for patients undergoing pain therapy. Methods of use comprising administering an effective amount and ratio of the opioid compounds to treat patients suffering from pain are also provided.

### BACKGROUND OF THE INVENTION

Opioid compounds remain key agents for the treatment of a wide variety of acute and chronic pain. The World Health Organization has recommended morphine as the analgesic of choice for the treatment of severe cancer pain. Additionally, morphine and related opioids are widely used to alleviate moderate to severe pain after surgery or trauma, or associated with medical illness. Patients with apparently similar pain states can have large differences in opioid dosing requirements. Factors that contribute to this variability include psychosocial status, type of pain (nociceptive, inflammatory, neuropathic or mixed) and its severity, concurrent medications, gender and other genetic aspects, and whether patients are opioid-naïve or tolerant.

Unfortunately, the effects produced by morphine and similar opioid compounds are associated with many undesirable side effects, all mediated through activation of the mu and other opioid receptors, and make them amenable to abuse. The undesirable side effects associated with the use of opioids include nausea and vomiting, drowsiness, dizziness, constipation, respiratory depression and bladder dysfunction. Also physical and psychological dependence leading to addiction and other diverse pathophysiological states can occur.

Further a major associated risk is that repeated daily administrations of morphine or morphine-like opioids will eventually induce significant tolerance to the therapeutic effects of the drug, as well as initiating some degree of physical dependence. Opioid tolerance is a phenomenon whereby chronic exposure to a drug diminishes its antinociceptive or analgesic effect, or creates the need for a higher dose to maintain its effect. The degree of tolerance and physical dependence will vary with the particular opioid employed, the correlation with morphine opioid receptor-selective opioids, the frequency of administration, and the quantity of opioid administered.

In a wide variety of clinical indications requiring prolonged use of opioids, tolerance induction and addiction are closely linked, with the development of physical and psychological dependence always a major concern. Addiction with physical dependence can be difficult to treat due to the effects of withdrawal associated with dependence. Another undesirable effect of opioid tolerance is that the higher opioid requirements of highly tolerant patients treated for pain increase the likelihood of unpleasant non-analgesic side effects due to greater circulating concentrations of opioids and potentially toxic opioid metabolites (Smith, M. T., Clin. Exp. Pharmacol. Physiol. 2000, 27, 524-528; Ross et al., Pain, 1997, 73, 151-157).

The opioid receptor is thought to have four receptor subtypes named mu (morphine receptor), sigma (the phencyclidine receptor), kappa (the ketocyclazocine receptor) and delta (the endorphin enkephalin receptor). The biochemical and cellular effects of morphine, including analgesia, are transduced through the mu opioid receptor (MOR), found in high concentrations within the central nervous system (CNS). The World Health Organization's guidelines for the management of chronic cancer pain recommend that clinicians reserve strong opioids, such as oxycodone and morphine, for the relief of moderate to severe cancer pain (World Health Organization, 1986). The guidelines also recommend that two strong opioids should not be co-administered, presumably because it is generally thought that all opioids exert their analgesic effects through the same receptor mechanisms in the central nervous system. However, studies by Maree Smith and co-workers have shown that the antinociceptive effects of structurally related oxycodone and morphine are differentially antagonized by nor-BNI (a -selective opioid antagonist) and naloxonazine (a selective -opioid receptor antagonist), indicating that they produce antinociception through different opioid receptor mechanisms (see Ross, F. B; Smith, M. T., Pain 1997, 73, 151-157). Furthermore, it has been found that co-administration to rats of sub-antinociceptive (also termed sub-analgesic) doses of oxycodone with morphine results in synergistic levels of antinociception (Ross et al., Pain 2000, 84, 421-428). It was found that animals that received the sub-antinociceptive doses of oxycodone and morphine were similar to vehicle injected control animals with respect to CNS side effects. Administration of equipotent-doses of either opioid alone resulted in sedation of the rats.

U.S. Pat. No. 6,310,072 to Smith et al. (incorporated herein in its entirety by reference) discloses analgesic compositions comprising a sub-analgesic dosage of a mu opioid agonist selected from the group consisting of morphine, fentanyl, sufentanil, alfentanil and hydromorphone, or a pharmaceutically acceptable salt thereof, and a sub-analgesic dosage of oxycodone which is a kappa-opioid agonist or a pharmaceutically acceptable salt thereof. Smith et al. disclose dosing regimens in terms of mg of therapeutic composition per kg of patient body weight over varying periods of time, but does not disclose a ratio of morphine and oxycodone that provide synergistic analgesia to human patients with reduction of opioid-related side effects. In particular, Smith et al. describe the administration of different combinations of morphine and oxycodone to male Sprague-Dawley and male Dark Agouti rats via intracerebroventricular (i.c.v.), intraperitoneal (i.p.) or subcutaneous (s.c.) administration, and the administration of a subanalgesic dose of morphine plus oxycodone (2.0 mg each) via intravenous administration. Smith et al. describes the use of a combination of sub-analgesic doses of morphine and oxycodone. Smith et al. defined the term ‘sub-analgesic dosage’ as a “dosage of a mu-opioid agonist solus or a kappa₂-opioid agonist solus which dosage does not result in the production of analgesia when administered to a human or antinociception when administered to a lower animal requiring alleviation of pain.” The sub-analgesic dosage ranges were defined in terms of commonly accepted lower thresholds for opioids that results in production of analgesia in human adults given by various routes of administration.

Bolan et al. (Journal of Pharmacology and Experimental Therapeutics, 2002, 303(2), 557-562) studied the combination of L-methadone and morphine with various other opioid receptor agonists in mice. It was reported that L-methadone exhibited synergy with morphine, morphine-6-β-glucuronide, codeine, and 6-acetylmorphine. However, both L-methadone and morphine displayed only additive effects with oxymorphone, oxycodone, fentanyl, alfentayl or meperidine.

Grach et al. (British Journal of Pharmacology, 2004) evaluated a combination of morphine and oxycodone in a ratio of 1 to 1 (by weight) dosed at 0.5 mg/kg of body weight versus morphine at 0.5 mg/kg of body weight and oxycodone at 0.5 mg/kg of body weight in a clinical pharmacodynamic setting with healthy subjects in a model of thermal pain. Comparisons of pain magnitude and side effects failed to show any significant differences between the three treatments. The authors concluded that at the doses and ratio tested, the co-administration of morphine and oxycodone did not produce synergistic antinociceptive effects or reduced CNS side effects in healthy humans in the cold pain model.

Clearly there is a need for a morphine and oxycodone-containing product suitable for treating a wide population of patients that provides both effective analgesia and reduced incidence and severity of adverse side effects.

### SUMMARY OF THE INVENTION

The present invention provides pharmaceutical compositions comprising an analgesic amount of morphine and an analgesic amount of oxycodone, or pharmaceutically acceptable salts thereof, in a ratio that provides fewer side effects when compared to doses of the individual components that provide equi-analgesia.

The pharmaceutical compositions of certain disclosed embodiments comprise a ratio of morphine to oxycodone of about 3 to about 2 by weight. The amounts of morphine and oxycodone are present in the pharmaceutical composition are both analgesic amounts. That is the amount of morphine and the amount of oxycodone present are each sufficient to induce analgesia. This ratio and amount of morphine to oxycodone provides the greatest relief from pain with improved side effect profiles.

In one embodiment, the invention provides an analgesic pharmaceutical composition for oral administration comprising morphine and oxycodone, or pharmaceutically acceptable salts thereof, in a ratio of from about 3 to about 2, morphine to oxycodone by weight, optionally in combination with a pharmaceutically acceptable carrier. In some embodiments, the pharmaceutical compositions of the invention comprise pharmaceutically acceptable salts of morphine and oxycodone including, but not limited to, salts formed with sulfuric or hydrochloric acid.

In some embodiments, the analgesic pharmaceutical composition for oral administration may comprise an analgesic amount of morphine and an analgesic amount of oxycodone in ratios of from about 3 to about 2 to about 1.25 to about 1, morphine to oxycodone by weight.

In one aspect, the pharmaceutical compositions of the invention are in the form of a solid oral dosage form. The oral dosage forms may be in an immediate release formulation or in the form of a controlled release formulation.

In some embodiments, the pharmaceutical compositions may be in the form of a tablet or capsule. In other embodiments, the pharmaceutical composition may be in a liquid oral dosage form.

In another embodiment, the invention provides pharmaceutical compositions suitable for intravenous or sub-cutaneous administration comprising morphine and oxycodone, or pharmaceutically acceptable salts thereof, in a ratio of about 3 to about 2, morphine to oxycodone by weight.

In some embodiments, the analgesic pharmaceutical composition for intravenous or sub-cutaneous administration may comprise an analgesic amount of morphine and an analgesic amount of oxycodone in ratios of from about 3 to about 2 to about 1.25 to about 1, morphine to oxycodone by weight.

Also provided is a method for the alleviation of pain in a patient comprising administering to the patient an analgesic pharmaceutical composition for oral administration comprising morphine and oxycodone, or pharmaceutically acceptable salts thereof, in a ratio of about 3 to about 2, morphine to oxycodone by weight, optionally in combination with a pharmaceutically acceptable carrier.

In some embodiments, the method for the alleviation of pain comprises orally administering to a patient a composition comprising an analgesic amount of morphine and an analgesic amount of oxycodone in ratios of about 3 to about 2 to about 1.25 to about 1, morphine to oxycodone by weight.

In other embodiments, the invention provides method for the alleviation of pain in a patient comprising administering to the patient an analgesic pharmaceutical composition for intravenous or sub-cutaneous administration comprising an analgesic amount of morphine and an analgesic amount of oxycodone in a ratio of about 3 to about 2, morphine to oxycodone by weight.

In some embodiments, the method for the alleviation of pain comprises administering intravenous or sub-cutaneous to a patient a composition comprising an analgesic amount of morphine and an analgesic amount of oxycodone in ratios of about 3 to about 2 to about 1.25 to about 1, morphine to oxycodone by weight.

Any suitable route of administration may be employed for providing a human or lower animal the composition of the invention. For example oral, rectal, parenteral, sublingual, buccal, intravenous, intraarticular, intramuscular, intradermal, subcutaneous, inhalational, intraocular, intraperitoneal, epidural, intracerebroventricular, transdermal and the like may be employed.

The present invention may be understood more readily by reference to the following detailed description of the specific embodiments included herein. However, although the present invention has been described with reference to specific details of certain embodiments thereof, it is not intended that such details should be regarded as limitations upon the scope of the invention. The entire text of the references mentioned herein are hereby incorporated in their entirety by reference.

### BRIEF DESCRIPTION OF THE DRAWING

FIG. 1 is a graph of the results of a test comparing the efficacy of a morphine/oxycodone combination (Q8003) with Percocet®.

FIG. 2 is two graphs showing the constipation ratings for a test comparing a low dose of morphine/oxycodone combination (Q8003), a flexible dose of morphine/oxycodone combination (Q8003) and Percocet®.

FIG. 3 is a graph of the results of a test comparing a low dose of morphine/oxycodone combination (Q8003), a flexible dose of morphine/oxycodone combination (Q8003) and Percocet® in terms of general activity, walking ability and sleep.

### DETAILED DESCRIPTION OF THE DISCLOSED EMBODIMENTS

The present invention comprises pharmaceutical compositions and methods comprising morphine and oxycodone for alleviating pain in a patient that provide an optimal analgesic efficacy while minimizing the incidence of undesired opioid side effects, particularly respiratory depression. The pharmaceutical compositions of the invention comprise an analgesic amount of morphine and an analgesic amount of oxycodone, or pharmaceutically acceptable salts thereof, in a ratio of about 3 to about 2 to about 1.25 to about 1, morphine to oxycodone by weight; preferably, about 3 to about 2, morphine to oxycodone by weight. These ratios and amounts of morphine and oxycodone provide the greatest relief from pain in humans at the lowest dose and with the best side effect profile. The optimal ratios and amounts of the two opioid compositions are determined in part by pharmacokinetic (PK) and pharmacodynamic (PD) profiles from patients or from patient groups treated with morphine and oxycodone combinations.

Where numeral number or range is modified by the term “about,” it will be understood to embrace somewhat larger or smaller values than the indicated value to account for experimental errors inherent in the measurement and variability between different methodologies for measuring the value, as will be apparent to one skilled in the art.

As discussed above, U.S. Pat. No. 6,310,072 to Smith et al., describes analgesic compositions comprising sub-analgesic doses of morphine and sub-analgesic doses of oxycodone. The administration of certain compositions comprising morphine and oxycodone to male Sprague-Dawley rats by intracerebroventricular (i.c.v.), or male Dark Agouti rats by intraperitoneal (i.p.) or subcutaneous (s.c.) administration resulted in synergistic analgesic efficacy. Further, it was reported that rats that received compositions of sub-analgesic doses of morphine and oxycodone approximating the ED₅₀ values of each of the morphine/oxycodone combinations via subcutaneous administration did not exhibit certain adverse side effects compared to rats that received doses approximating the ED₅₀ of morphine or oxycodone alone.

Notwithstanding the efficacy of compositions comprising sub-analgesic doses of morphine and sub-analgesic doses of oxycodone demonstrated in rats as described in U.S. Pat. No. 6,310,072, it is known that the oxycodone and morphine have different intrinsic antinociceptive potencies in rats and humans (see for example Ross et al., Pain 73 (1997), 151-157), and synergistic efficacy in one animal model is not predictive of efficacy in human patients. Additionally, it has been reported that certain combinations of morphine and oxycodone in another animal model did not result in synergistic efficacy and exhibited only additive effects (Bolan et al., The Journal of Pharmacology and Experimental Therapeutics, 2002, 303(2), 557). For example, Bolan et al. reported that morphine in combination with L-methadone exhibited synergistic analgesic efficacy in male CD-1 mice, but that morphine in combination with oxymorphone, oxycodone, fentanyl, alfentanyl or meperidine displayed only additive effects.

In contrast to the synergistic efficacy of combinations of sub-analgesic doses of morphine and sub-analgesic doses of oxycodone described in the U.S. Pat. No. 6,310,072 in male Sprague-Dawley and male Dark Agouti rats, Grach and co-workers reported that the combination of analgesic doses of morphine and analgesic doses of oxycodone in a 1 to 1 weight ratio administered orally to humans has been reported to not yield a synergistic analgesic efficacy in a cold pain model (Grach et al., British Journal of Clinical Pharmacology, 2004). Administration of 0.25 mg/kg morphine sulfate in combination with 0.25 mg/kg oxycodone hydrochloride via oral administration compared 0.5 mg/kg morphine sulfate alone or 0.5 mg/kg oxycodone hydrochloride alone failed to shown synergistic analgesic behavior when administered to human volunteers exposed to an experimental model of cold pressor test (CPT). These reports highlight the complex and unpredictable nature of the cross-reactivity of opioid receptor agonists.

The ratios of morphine to oxycodone of the present invention relate to the weight ratio of the parent compounds in the neutral state. However, it will be apparent to those skilled in the art that the compositions of the invention may comprise pharmaceutically acceptable salts of the compounds as long as the salts are present in an amount that corresponds to the desired weight ratio of the parent compounds. The pharmaceutically acceptable salts of morphine and oxycodone may be prepared with any pharmaceutically acceptable acid including, but not limited to, sulfuric acid and hydrochloric acid.

The pharmaceutical compositions may be in the form of solid or liquid dosage forms including, by way of example and not limitation, tablets, capsules, dispersions, suspensions, injections, solutions, syrups, troches, capsules, suppositories, aerosols, transdermal patches and the like. These dosage forms may also include compositions implanted in a patient that are designed to provide sustained release of the active agents. Controlled release of the opioids may be affected by incorporating the opioids into, by way of example and not limitation, hydrophobic polymers, including acrylic resins, waxes, higher aliphatic alcohols, polylactic and polyglycolic acids and certain cellulose derivatives, such as hydroxypropylmethyl cellulose. In addition, the controlled release may be affected by using other polymer matrices, liposomes and/or microspheres.

Pharmaceutically-acceptable carriers for systemic administration also may be incorporated into the compositions of the present invention. By “pharmaceutically-acceptable carrier” is meant a solid or liquid filler, diluent or encapsulating substance which may be safely used in systemic administration. Depending upon the particular route of administration, a variety of pharmaceutically-acceptable carriers well known in the art may be used. These carriers include, by way of example and not limitation, sugars, starches, cellulose and its derivatives, malt, gelatin, talc, calcium sulfate, vegetable oils, synthetic oils, polyols, alginic acid, phosphate buffered solutions, emulsifiers, isotonic saline, and pyrogen-free water.

In one embodiment, the invention provides an analgesic pharmaceutical composition for oral administration comprising an analgesic amount of morphine and an analgesic amount of oxycodone, or pharmaceutically acceptable salts thereof, in a ratio of about 3 to about 2 to about 1.25 to about 1, morphine to oxycodone by weight. In other embodiments, the invention provides pharmaceutical compositions comprising morphine and oxycodone, or pharmaceutically acceptable salts thereof, in a ratio of about 3 to about 2, morphine to oxycodone by weight. In other embodiments, the invention provides pharmaceutical compositions comprising morphine and oxycodone, or pharmaceutically acceptable salts thereof, in a ratio of about 1.25 to about 1, morphine to oxycodone by weight.

In one embodiment, the compositions of the present invention may be administered by intravenous or sub-cutaneous routes. The sterile injectable preparation may be a sterile injectable solution or suspension in a non-toxic parenterally-acceptable diluent or carrier known in the art for intravenous or sub-cutaneous administration, for example as a solution in water, physiological saline, Ringer's solution, or phosphate buffered saline (PBS), or in suitable non-aqueous carriers.

In a particular embodiment, the invention provides a composition comprising an analgesic amount of morphine and an analgesic amount of oxycodone, or pharmaceutically acceptable salts thereof, suitable for intravenous or sub-cutaneous administration, wherein the morphine and oxycodone are in a ratio of about 3 to about 2 to about 1.25 to about 1, morphine to oxycodone by weight. In other embodiments, the invention provides pharmaceutical compositions comprising an analgesic amount of morphine and an analgesic amount of oxycodone, or pharmaceutically acceptable salts thereof, suitable for intravenous or sub-cutaneous administration in a ratio of about 3 to about 2, morphine to oxycodone by weight. In still other embodiments, the invention provides pharmaceutical compositions comprising an analgesic amount of morphine and an analgesic amount of oxycodone, or pharmaceutically acceptable salts thereof, suitable for intravenous or sub-cutaneous administration in a ratio of about 1.25 to about 1, morphine to oxycodone by weight.

In another aspect of the invention, pharmaceutical compositions comprising morphine and oxycodone in formulations that are suitable for oral administration are provided. Pharmaceutical compositions suitable for oral administration may be in the form of solid oral dosage forms or liquid oral dosage forms and may include pharmaceutically acceptable carriers and excipients known in the art. Suitable vehicles and their formulation are described, for example, in Remington: The Science and Practice of Pharmacy, 21st ed, Lippincott Williams & Wilkins (2005).

The concentration of morphine and oxycodone in the blood stream will depend on the amount of compound administered in the composition as well as the method of administration and the specific formulation used. For example, it is well known in the art that administration of morphine and oxycodone by intravenous injection typically results in a significant concentration of each compound in the blood stream substantially immediately after administration (without delay), whereas formulations adapted for oral administration of morphine and oxycodone will typically achieve effective concentrations in the blood stream later than intravenous administration and at different concentrations depending on oral availability of the compounds. Further, the means of administration of the compounds may result in different inactivation and excretion rates of morphine and oxycodone when administered in a combination. Therefore, it will be apparent to one of skill in the art that the absolute and relative amounts of morphine and oxycodone administered to patients via oral administration to achieve a synergistic efficacy with a lower incidence of adverse side effects will differ from the amounts of drugs required for intravenous administration or other routes of administration.

The pharmaceutical compositions for oral administration may be in the form of immediate release or controlled release formulations. Pharmaceutical compositions comprising immediate release or controlled release formulations of opioid drugs are well known in the art. For example, OXYIR® is an immediate release formulation of oxycodone and OXYCONTIN® in a controlled release formulation of oxycodone, which provides controlled delivery of oxycodone over 12 hours. Similarly, MS CONTIN® is a controlled release formulation of morphine sulfate and MSIR® is an immediate release formulation of morphine sulfate.

Immediate release formulations of pharmaceutical compositions will typically comprise ingredients that break down quickly after administration to release the active components. In some embodiments, the pharmaceutical compositions of the invention in immediate release formulations will exhibit a Tₘₐₓ, the time required for a compound to reach its maximum concentration in circulation (Cₘₐₓ), of from about 10 minutes to about 2 hours after ingestion. In other embodiments, the Tₘₐₓ will be from about 10 minutes to about 1 hour, about 10 minutes to about 30 minutes or about 10 minutes to about 45 minutes.

The pharmaceutical compositions in controlled release or sustained release formulations will typically exhibit longer Tₘₐₓ times and display significant concentrations in circulation for longer periods of time. In some embodiments, the Tₘₐₓ of controlled release formulations of the invention will be from about 1 hour to about 4 hours, from about 1 hour to about 3 hours, from about 1 hour to about 2.5 hours or from about 1 hour to about 2 hours.

The controlled release formulation of morphine and oxycodone will be released at a slower rate and over a longer period of time. For example, in some embodiments, the controlled release formulation of morphine and oxycodone will release effective amounts of a mixture of morphine and oxycodone over 12 hours. In other embodiments, the controlled release formulation will release effective amounts of morphine and oxycodone over 4 hours or over 8 hours. In still other embodiments, the controlled release formulation will release effective amounts of morphine and oxycodone over 15, 18, 24 or 30 hours.

The absolute amounts of morphine and oxycodone present in the pharmaceutical compositions of the invention or administered in the methods of the invention are each analgesic amounts; i.e., an amount of morphine sufficient to provide analgesia in the patient if administered alone and an amount of oxycodone sufficient to provide analgesia in the patient if administered alone. Producing analgesia in each patient is different and therefore will be determined for a particular patient based on the specific needs of the patient and the type of pain being treated. The specific dose will depend upon a variety of patient-specific factors, including, but not limited to, the age, the body weight, general health, sex, diet, time of administration, rate of excretion, and the judgment of the treating physician and the severity of the condition that requires analgesic treatment. Further, the amount of the active compounds that may be combined with the carrier materials to produce a single dosage form will vary depending upon the patient treated, the purpose of treatment, the pain state and whether the patient is opioid-naïve or has developed tolerance. The particular mode of administration will also affect the dose of the compound given to a patient.

Table 1 below list amounts of morphine and amounts of oxycodone that are generally required to produce analgesia in an average human patient. Of course, individual dosing requirements vary considerably based on each patient's age, body weight, severity of pain, and medical and analgesic history. In elderly and debilitated patients and patients with impaired respiratory and renal functions, the initial dose should be one-half of the usual recommended dose.

TABLE 1

| Opioid | Used as the first opioid analgesic | Patients currently receiving opioids |
|---|---|---|
| Morphine IR, | Initial dose: | Initial dose: |
| oral tablets | Acute pain: 10 mg Q4 h as needed | The initial dose should be the oral |
| | Chronic pain: 10 mg Q4 h | morphine equivalent dose of the |
| | Maintenance dose: | current opioids. It should be |
| | Varies. The maintenance dose should | calculated using the appropriate |
| | be individualized. The initial dose | opioid conversion factor. |
| | should be titrated (up or down) to the | Maintenance dose: |
| | lowest dose that maintains the patient | Varies. The maintenance dose should |
| | free of pain or with minimal pain | be individualized. The initial dose |
| | (maintenance dose). | should be titrated (up or down) to the |
| | | lowest dose that maintains the patient |
| | | free of pain or with minimal pain |
| | | (maintenance dose). |
| Morphine | SR/ER/CR oral morphine is not | Initial dose: |
| SR/ER/CR, | recommended as initial opioid | The initial dose should be the oral |
| oral tablets or | analgesic. It is advisable to start | morphine equivalent dose of the |
| capsules | treatment with IR morphine. | current opioids. It should be |
| | If SR/ER is chosen as the first opioid | calculated using the appropriate |
| | analgesic: | opioid conversion factor. |
| | Initial dose: | Maintenance dose: |
| | Initial total daily dose should be about | Varies. The maintenance dose should |
| | 30 mg (e.g. 10 mg tid, 15 mg bid, or | be individualized. The initial dose |
| | 30 mg qd) | should be titrated to the lowest dose |
| | Maintenance dose: | that maintains the patient free of pain |
| | Varies. The initial dose should be | or with minimal pain (maintenance |
| | titrated to the maintenance dose in | dose). |
| | increments not greater than 30 mg | |
| | every four days. | |

| Morphine | Initial dose should be 2 to 10 mg/70 kg of body weight. | |
|---|---|---|
| Sulfate | | |
| Injection, iv | | |
| Morphine | 2:1 or 3:1 conversion from oral morphine dose: e.g. if a total oral morphine | |
| Sulfate | dose is 240 mg/day, the equivalent SC dose would be 120 mg/day (2:1 ratio)¹². | |
| Injection, sc | | |
| Oxycodone IR | The usual dose is 5 mg every 6 hours, PRN. | |
| oral capsules | | |
| Oxycodone CR | Initial dose: | Initial dose: |
| oral tablets | Initial dose should be about 10 mg, | The initial dose should be converted |
| | q12 h | from the dose of current opioids. It |
| | Maintenance dose: | should be calculated using the |
| | Varies. The initial dose should be | appropriate conversion factor⁹. |
| | titrated to the maintenance dose in | Maintenance dose: |
| | increments of 25% to 50% of the | Varies. The maintenance dose should |
| | current dose. | be individualized. The initial dose |
| | | should be titrated to the lowest dose |
| | | that maintains the patient free of pain |
| | | or with minimal pain (maintenance |
| | | dose). |

The upper dose range of either morphine or oxycodone is that amount that will not be fatal to the patient. The Avinza® package insert states that the daily dose of Avinza (morphine sulfate, ER oral) must be limited to a maximum of 1600 mg/day. OxyContin's package insert indicates that OxyContin 60, 80, and 160 mg tablets or a single dose greater than 40 mg are for use in opioid-tolerant patients only. A single daily dose greater than 40 mg or total daily doses greater than 80 mg may cause fatal respiratory depression when administrated to patients who are not tolerant to the respiratory depressive effect of opioids. Other package inserts indicate that attention should be given to special safety issues associated with oxycodone HCL ER doses at or greater than 160 mg, q12h.

In some embodiments of the invention, the analgesic dose of the morphine component, or pharmaceutically acceptable salts thereof, in the pharmaceutical compositions in accordance with the present invention or methods of the present invention for a näive human adult through oral or rectal administration and in immediate release form are 3 mg or more; preferably, 6 mg or more; especially, 12 mg or more; and, most especially, 18 mg or more, every four hours. For non-näive human adults through oral administration in immediate release form, the dose of the morphine component will be higher.

In some embodiments of the invention, the analgesic dose of the morphine component, or pharmaceutically acceptable salts thereof, in the pharmaceutical compositions in accordance with the present invention or methods of the present invention for a näive human adult through oral administration and in immediate release form are about 3 mg every four hours up to a maximum dose of about 1,600 mg per day; preferably, about 6 mg to about 200 mg every four hours; especially, about 12 mg to about 200 mg every four hours; most especially, about 18 to about 200 mg every four hours. For non-näive human adults through oral administration in immediate release form, the dose of the morphine component will to be higher, such as up to about 1,600 mg per day.

In some embodiments of the invention, the analgesic dose of the morphine component, or pharmaceutically acceptable salts thereof, in the pharmaceutical compositions in accordance with the present invention or methods of the present invention for a näive human adult through oral administration and in controlled release or sustained release form are about 10 mg to about 1600 mg per day. For non-näive human adults through oral administration in controlled release or sustained release form, the dose of the morphine component will to be higher, such as up to about 1600 mg per day.

In some embodiments of the invention, the analgesic dose of the morphine component, or pharmaceutically acceptable salts thereof, in the pharmaceutical compositions in accordance with the present invention or methods of the present invention for an opioid näive human adult in intravenous or sub-cutaneous form or through intravenous or sub-cutaneous administration are about 2 mg to about 10 mg per 70 kg of body weight up to a maximum daily dose of 800 mg per day. For non-näive human adults through intravenous or sub-cutaneous administration, the dose of the morphine component will to be higher, such as up to about 800 mg.

In some embodiments of the invention, the analgesic dose of the oxycodone component, or pharmaceutically acceptable salts thereof, in the pharmaceutical compositions in accordance with the present invention or methods of the present invention for a näive human adult through oral or rectal administration and in immediate release form are 2 mg or more; preferably, 4 mg or more; especially, 8 mg or more; and, most especially, 12 mg or more, every four hours. For non-näive human adults through oral administration in immediate release form, the dose of the morphine component will be higher.

In some embodiments of the invention, the analgesic dose of the oxycodone component, or pharmaceutically acceptable salts thereof, in the pharmaceutical compositions in accordance with the present invention or methods of the present invention for a näive human adult through oral administration and in immediate release form are about 2 mg every four hours up to a maximum dose of about 320 mg per day; preferably, about 4 mg every four hours up to a maximum dose of about 320 mg per day; especially, about 8 mg every four hours up to a maximum dose of about 320 mg per day; most especially, about 12 mg every four hours up to a maximum dose of about 320 mg per day. For non-näive human adults through oral administration in immediate release form, the dose of the morphine component will to be higher, such as up to about 320 mg per day.

In some embodiments of the invention, the analgesic dose of the morphine component, or pharmaceutically acceptable salts thereof, in the pharmaceutical compositions in accordance with the present invention or methods of the present invention for a näive human adult through oral administration and in controlled release or sustained release form are about 10 mg every 12 hours up to a maximum daily does of about 400 mg per day. For non-näive human adults through oral administration in controlled release or sustained release form, the dose of the morphine component will to be higher, such as up to about 400 mg per day.

In some embodiments of the invention, the analgesic dose of the oxycodone component, or pharmaceutically acceptable salts thereof, in the pharmaceutical compositions in accordance with the present invention or methods of the present invention for an opioid näive human adult in intravenous or sub-cutaneous form or through intravenous or sub-cutaneous administration are about 2 mg to about 10 mg per 70 kg of body weight. For non-näive human adults through intravenous or sub-cutaneous administration, the dose of the morphine component will to be higher, such as up to about 200 mg per day.

Following extensive studies investigating the varying impacts of administering therapeutic opioid compositions to human patients in order to alleviate pain symptoms while minimizing opioid-related adverse side effects, the present inventors determined that the preferred range of ratios of morphine and oxycodone in dual opioid compositions is about 3 to 2 to about 1.25 to 1, morphine to oxycodone by weight; especially about 3 to 2 morphine to oxycodone by weight.

The present invention will be understood more readily by reference to the following examples, which are provided by way of illustration and are not intended to be limiting of the invention.

### Example 1

### Efficacy and Safety Study of Products Containing Morphine and Oxycodone in a Ratio of 3 to 2 (by Weight) in Acute Pain

This double-blind, ascending cohort, parallel treatment study evaluated analgesia and safety measures in 5 groups of patients with moderate to severe pain (numerical pain rating scale score, range 0-10, study inclusion score of at least 4) following bunionectomy surgery, a procedure that involves manipulation of metatarsal foot bone. Once each of the 256 patients had sufficient pain following surgery, they were randomized to morphine/oxycodone or placebo and received dosing for up to the next 48 hrs. The dosing schedule was flexible in that the inventors wanted to determine what dosing intervals were preferred (and the amount of drug received) by patients as a function of unit dose strengths of products morphine and oxycodone in the ratio of 3 to 2 by weight. Dosages administered were 3/2 mg (3 mg of morphine and two mg of oxycodone), 6/4 mg (6 mg of morphine and 4 mg of oxycodone), 12/8 mg (12 mg of morphine and 8 mg of oxycodone) and 18/12 mg (18 mg morphine and 12 mg of oxycodone) or 0/0 mg (placebo). Patients were also allowed rescue analgesia (600 mg ibuprofen), but following ibuprofen dosing pain data was censored for a period of time. In addition to the pain ratings, a patient rating of overall clinical satisfaction with study medication at the end of dosing was utilized. A total of about 50-60 patients entered into each of the 5 treatment arms. Also collected were plasma samples for post-study measurement of blood levels of morphine and of oxycodone (3 samples per patient over the 48 hr study period).

Using the data on blood plasma levels of morphine and oxycodone measured at three time points (sparse sampling approach) during the 48 hr dosing period, a pharmacokinetic model was constructed in which the full curve of plasma morphine and of oxycodone levels over a 48 hr period for each patient was generated. The model itself used PK data from prior single dose and repeat dose phase 1 studies of morphine/oxycodone products. Also a definition of patients that received superior pain relief (termed ‘superior responders’) and of patients that received adequate pain relief (termed ‘adequate responders’) was provided using the efficacy data obtained from the Study. For the PK/PD analysis, a patient with superior pain relief was defined as a patient who at termination of treatment had at least a 30% reduction in pain intensity from baseline and/or a global rating of good to excellent satisfaction with the treatment effect.

Using a Global Score of Satisfaction of pain control (5 point scale where 0=poor, 1=fair, 2=good, 3=very good, and 4=excellent)) it was found that all doses of the combination product gave statistically and clinically superior results to the placebo. Table 2 below shows the percentages of patients that had good-excellent Global Score responses.

TABLE 2
Pain Control of MoxDuo ™ Products verses Placebo

| | Percent Patients with |
|---|---|
| | Good to Excellent |
| Product administered | Global Scores |
| 0/0 mg - Placebo | 13% |
| 3/2 mg | 44% |
| 6/4 mg | 43% |
| 12/8 mg | 58% |
| 18/12 mg | 41% |

Further comparison of the extent of pain reduction over a 48 hr period (SPID48 [sum of pain intensity differences]; i.e., the area under the curve of the changes in pain from baseline for the 48 hr period following the first dose of study medication) for the 3/2 product dosed on average once every 4 hrs versus placebo showed mean SPID48 scores of 34 and −10, respectively. These results support the conclusion that all doses of the combination morphine/oxycodone product gave superior pain relief to placebo.

The PK/PD data for patients with superior responders versus partial responders (data pooled across all dose levels) was analyzed. Table 3 below shows key results of PK parameters for superior responders (n=129) versus adequate responders (n=64) patients who received the morphine/oxycodone containing products (placebo data are omitted). The Table summarizes the results for adequate responders and superior responder patients for several PK parameters.

TABLE 3
Summary of PK/PD data for superior responders versus adequate responders (data pooled
across all dose levels).

| | | Mean | | | |
|---|---|---|---|---|---|
| | Mean Partial | Superior | | | |
| | Responders | Responders | | | Mean: All |
| | (n = 64 pts) | (n = 129) | t-test | p-value | Patients |
| Median Ratio of | 0.4421 | 0.2793 | 4.80 | <0.001 | 0.3333 |
| Plasma Levels of | | | | | |
| Morphine to | | | | | |
| Oxycodone | | | | | |
| Ratio of AUC | 0.2478 | 0.2347 | 1.57 | 0.119 | 0.2391 |
| Morphine to AUC | | | | | |
| Oxycodone | | | | | |
| Morphine Cmax (nM) | 10.09 | 10.88 | 0.97 | NS | 10.63 |
| Oxycodone Cmax | 44.18 | 49.08 | 1.44 | 0.152 | 47.45 |
| (nM) | | | | | |
| Morphine AUC | 217.47 | 259.47 | 1.61 | 0.108 | 245.54 |
| (nM*hr) | | | | | |
| Oxycodone AUC | 908.88 | 1144.50 | 2.18 | 0.030 | 1066.37 |
| (nM*hr) | | | | | |

*the median ratio was determined for each patient across all time points. The t-tests compared the mean of the median ratios for superior responders vs. adequate responders.

All combination products tested gave superior pain relief compared to patients taking a placebo. Also, the only parameter that showed a pronounced statistically significant difference between superior responders and partial responders was the ratio of the mean plasma level of morphine to oxycodone. The lower ratios of the mean plasma levels of morphine to oxycodone in the responder group means that patients with a higher proportion of morphine to oxycodone in the blood tended to have a better response. The superior responders had an oxycodone to morphine ratio that was about 1.6 times that of the partial responders. The ratio of the areas under the curve (AUCs), which takes into account the magnitude of the plasma concentrations at each time point, showed a non-significant trend in the same direction, for example, higher proportions of morphine to oxycodone gave a better effect. This non-significant trend is consistent with the observation that absolute amounts of drug in blood were not strongly related to outcome.

Based on the investigations detailed herein, it is projected that formulations containing morphine plus oxycodone in a ratio of 3 to 2 (by weight) produce a plasma ratio of morphine to oxycodone level of approximately 0.33 or less during repeat dosing produces, and, thus, yield a significantly better therapeutic effect compared to formulations containing morphine plus oxycodone in a ratio of less than 3 to 2 by weight.

### Example 2

### Comparison of a Product Containing Morphine and Oxycodone in the Ratio of 3 to 2 vs. Morphine vs. Oxycodone in the Treatment of Acute Pain

The purpose of this study was to compare the efficacy and opioid related adverse events of products containing analgesic doses of morphine and oxycodone in the ratio of 3 to 2 by weight with the individual components.

The study was a double-blind, randomized, multicenter, 48 hr treatment duration, bunionectomy study. The fixed dose treatment arms (q6h) were (i) products containing morphine and oxycodone as follows: 12/8 mg (12 mg morphine and 8 mg oxycodone) and 6/4 mg (6 mg morphine and 4 mg oxycodone), and (ii) 4 products containing the components alone as follows: morphine 12 mg, morphine 6 mg, oxycodone 8 mg and oxycodone 4 mg. Also, ibuprofen rescue medication was 400 mg and was limited to a total dose of 3200 mg per 24 hrs. A total of 197 patients were enrolled in the study. A pain relief equivalency ratio of 1.5 parts of morphine is equivalent to one part of oxycodone was used in determining ‘morphine equivalents’ for comparison purposes (see Table 4). Efficacy and side effect reduction were measured in the study.

TABLE 4
Morphine Equivalent Doses of Product Used in the Study

| Product Containing | | |
|---|---|---|
| Morphine to Oxycodone | | |
| in the Ratio of 3 to 2 | Morphine- | Oxycodone- |
| (by weight) | Equivalent Dose | Equivalent Dose |
| Product: 6/4 mg | 12 mg | 8 mg |
| Product: 12/8 mg | 24 mg* | 16 mg* |

*These doses were not studied in the current study

Using pain reduction over a 48 hr period (SPID48: area under the curve of the changes in pain from baseline for the 48 hr period following the first dose of study medication) the results show that all doses including the low doses of morphine (6 mg) and oxycodone (4 mg) alone all gave pain control and are judged analgesic doses.

TABLE 5
Pain Reduction of Various MoxDuo ™ Products

| Product | |
|---|---|
| administered | Mean SPID48 Score |
| 12/8 mg | 148 |
| 6/4 mg | 97 |
| Morphine 12 mg | 106 |
| Morphine 6 mg | 51 |
| Oxycodone 8 mg | 99 |
| Oxycodone 4 mg | 60 |

The key safety results from the study are shown in Table 6 (opioid related adverse events). When comparing equi-analgesic doses (i.e., doses with of the same morphine equivalents) the opioid adverse event (AE) incidence for the 6/4 mg product (which is the morphine equivalent dose of 12 mg morphine or of 8 mg oxycodone) had much lower rates of moderate to severe nausea, vomiting, dizziness and constipation compared to the monotherapy groups (Table 6). There were 20-75% decreases in most of these event rates in the 6/4 mg product. Also the overall tolerance as measured by the number of patients dropping out during the study was better in the combination product compared to its individual components.

TABLE 6
Comparative AE Percents for the Most Common
Opioid Events at Morphine Equivalent Doses

| | Product | | |
|---|---|---|---|
| | Morphine & | Morphine | Oxycodone |
| | Oxycodone 6/4 mg | 12 mg | 8 mg |
| Morphine Equivalent of | 12 mg | 12 mg | 12 mg |
| Product | | | |
| Number of Patients per | 32 | 29 | 34 |
| Group | | | |
| Adverse Events (%) | | | |
| Nausea | 44 | 59 | 50 |
| mild/mod/severe | | | |
| Nausea - | 9 | 28 | 26 |
| moderate/severe | | | |
| Vomiting | 6 | 21 | 21 |
| mild/mod/severe | | | |
| Vomiting - | 3 | 21 | 21 |
| moderate/severe | | | |
| Dizziness | 13 | 31 | 24 |
| mild/mod/severe | | | |
| Dizziness - | 3 | 7 | 12 |
| moderate/severe | | | |
| Constipation | 3 | 10 | 6 |
| mild/mod/severe | | | |
| Constipation - | 0 | 0 | 0 |
| moderate/severe | | | |
| Dropouts (%) | 3 | 7 | 12 |

The results of the study show that patients treated with the 6/4 mg combination morphine/oxycodone product experienced significantly fewer adverse events than those patients that were treated with equi-analgesic doses of the monotherapy; i.e., equivalent does of either morphine or oxycodone.

### Example 3

### Comparison of a Product Containing Morphine and Oxycodone in the Ratio of 3 to 2 vs. a Percocet, a Marketed Combination Product Containing Oxycodone 5 mg and Acetaminophen 325 mg in the Treatment of Acute Pain

The purpose of the study was to compare the efficacy and opioid related adverse events to determine the potential advantages of the morphine plus oxycodone product to that of a widely used marketed analgesic combination drug, Percocet (oxycodone plus acetaminophen).

The study was an open label, randomized, multicenter, 48 hr treatment duration with dosing given q4-6 hr (but not more than 4 times per day), study in patients with moderate to severe pain following primary unilateral total knee arthroplasty. The doses of the combination morphine/oxycodone product (MoxDuo™) were flexible, depending upon analgesic need and tolerability. Dosage for one group was 3/2 mg (3 mg of morphine and 2 mg of oxycodone); dosage for another group was a flexible dose that ranged from 12/8 mg (12 mg of morphine and 8 mg of oxycodone) to 18/12 mg (18 mg of morphine and 12 mg of oxycodone). The Percocet tablets were 5 mg/325 mg (oxycodone/acetaminophen) and 1-2 tablets were permitted per dose, depending on need and tolerability. Pain reduction over 48 hrs was assessed by periodically evaluating the changes in pain intensity from baseline over 48 hrs (SPID48; area under the curve of the changes in pain intensities). The pain intensity scores were measured using the Numerical Pain Rating (NPRS) scale, range 0 to 10 (no pain to most intense pain imaginable). Also, in this trial acetaminophen rescue medication of up to 1000 mg q-6h was allowed in the opioid combination group. A total of 43 patients were enrolled in the study.

Table 7 shows patient disposition and mean dosing.

TABLE 7

| | MoxDuo ™ | MoxDuo ™ | |
|---|---|---|---|
| | Flexible Dose | 3/2 mg | Percocet ® |
| Randomized | N = 14 | N = 14 | N = 15 |
| Discontinued | 3 (23%) | 5 (39%) | 3 (20%) |
| Median Hrs | 43.3 | 32.2 | 37.1 |
| on Study Drug | | | |
| Mean mg per | 14.4/9.6 | 3/2 | 7.8/507 |
| Dose | (morphine/ | (morphine/ | (oxycodone/ |
| | oxycodone) | oxycodone) | acetaminophen) |

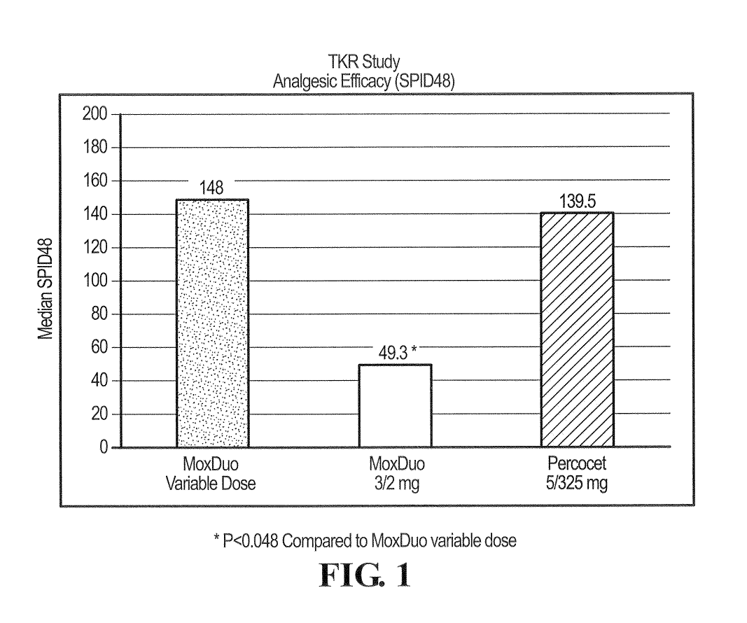

The key efficacy results are shown in FIG. 1. These flexible doses of Q8003 and Percocet® produced comparable pain reduction, as shown in the SPID48 measures in FIG. 1.

Table 8 shows the moderate to severe adverse events reported by the patients participating in this study.

TABLE 8
Comparative AE Percents

| | Q8003 | Percocet ^{®} |
|---|---|---|
| Adverse Event | N = 14 | N = 15 |
| Any GI AE | 14% | 47% |
| Nausea | 0% | 27% |
| Emesis | 0% | 20% |
| Constipation | 7% | 13% |
| Hypotension | 0% | 13% |
| Somnolence | 0% | 0% |
| Headache | 0% | 0% |
| Dizziness | 0% | 0% |

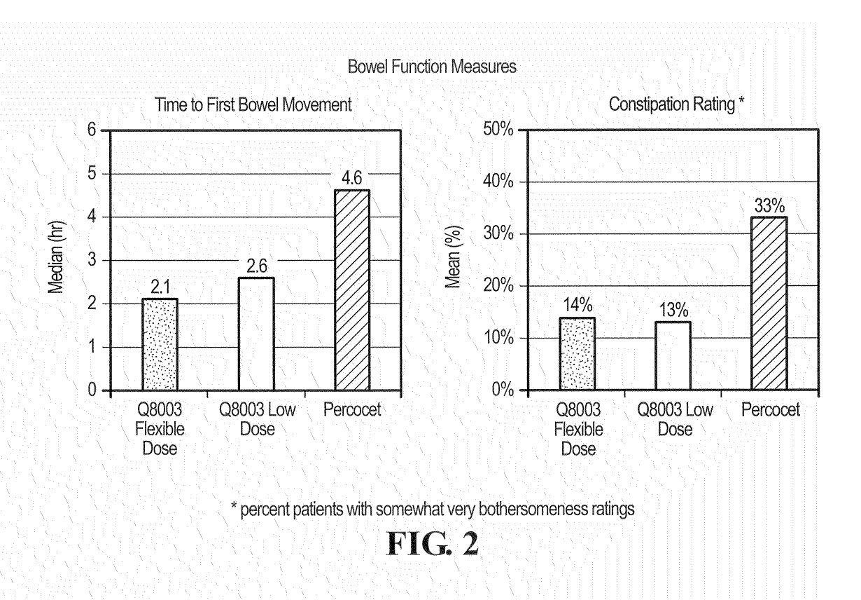

When comparing these equi-analgesic doses, the spontaneously reported opioid adverse event (AE) incidences were appreciably different, as shown in Table 8. There were substantially lower rates of moderate to severe gastrointestinal (GI) AEs, including less nausea; emesis and constipation in the Q8003 treated subjects, despite the fact that they received 3 times the morphine equivalent dose as the Percocet group. Both treatment groups had no moderate-severe instances of somnolence, headache or dizziness. FIG. 2 demonstrates the percentage of patients that reported bothersome instances of constipation during the 48 hr dosing period. There was less impairment of bowel function in Q8003 treated patients, despite their receiving higher morphine equivalent doses than occurred in the Percocet treated patients.

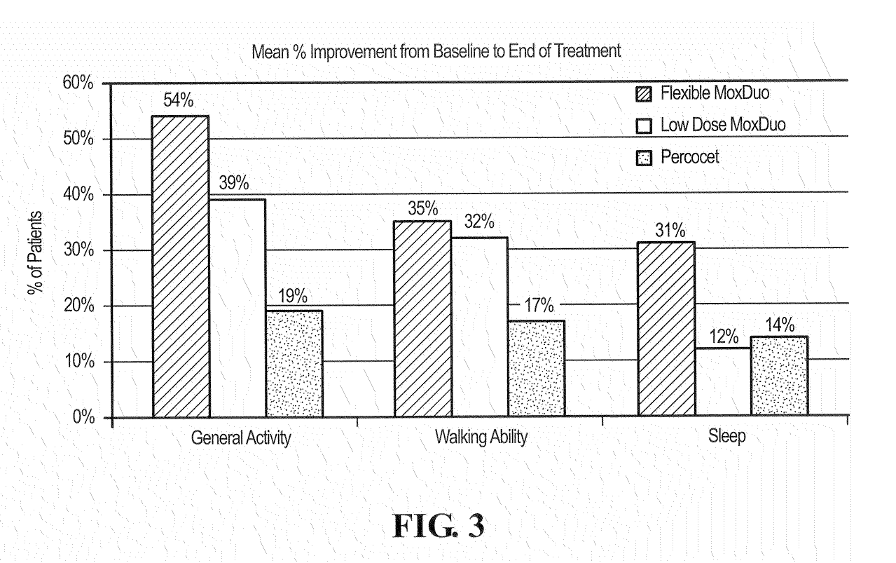

FIG. 3 demonstrates that both the Low Dose MoxDuo™ group and the Variable Dose MoxDuo™ group experienced better general activity, better walking ability and better sleeping ability than the Percocet® group.

While the present invention has been illustrated and described in relation to certain potentially preferred embodiments and practices, it is to be understood that the illustrated and described embodiments and practices are illustrative only and that the present invention includes such embodiments but is not limited thereto. Rather, it is fully contemplated that modifications and variations to the present invention will no doubt occur to those of skill in the art upon reading the above description and/or through practice of the invention. It is therefore intended that the present invention shall extend to all such modifications and variations as may incorporate the broad principles of the present invention within the full scope thereof as set forth in the appended claims.

What is claimed is:

1. An analgesic pharmaceutical composition comprising morphine and oxycodone, or pharmaceutically acceptable salts thereof, in a ratio of from about 3 to 2 to about 1.25 to 1, morphine to oxycodone by weight, optionally in combination with a pharmaceutically acceptable carrier, wherein the morphine and oxycodone are each present in analgesic amounts.

2. The pharmaceutical composition of claim 1, wherein the composition comprises pharmaceutically acceptable salts of morphine and oxycodone.

3. The pharmaceutical composition of claim 1, wherein the ratio of morphine to oxycodone is about 3 to 2.

4. The pharmaceutical composition of claim 1, wherein the composition is in the form of a solid oral dosage form.

5. The pharmaceutical composition of claim 4, wherein the dosage form comprises an immediate release formulation.

6. The pharmaceutical composition of claim 4, wherein the dosage form comprises a controlled release formulation.

7. The pharmaceutical composition of claim 4, wherein the dosage form is a tablet or capsule.

8. The pharmaceutical composition of claim 1, wherein the composition is in a liquid oral dosage form.

9. The pharmaceutical composition of claim 1, wherein the composition is in a liquid intravenous or sub-cutaneous dosage form.

10. The pharmaceutical composition of claim 1, wherein the morphine and oxycodone are present in amounts of about 3 mg morphine and about 2 mg oxycodone, about 6 mg morphine and about 4 mg oxycodone, about 12 mg morphine and about 8 mg oxycodone or about 18 mg morphine and about 12 mg oxycodone.

11. A method for the alleviation of pain in a patient comprising administering to the patient the pharmaceutical composition of claim 1.

12. The method composition of claim 11, wherein the ratio of morphine to oxycodone is about 3 to 2.

13. A method for the alleviation of pain in a patient comprising administering to the patient a pharmaceutical composition comprising morphine and oxycodone, or pharmaceutically acceptable salts thereof, in a ratio of from about 3 to 2 to about 1.25 to 1, morphine to oxycodone by weight, optionally in combination with a pharmaceutically acceptable carrier, wherein the morphine and oxycodone are each present in analgesic amounts.

14. The pharmaceutical composition of claim 13, wherein the composition comprises pharmaceutically acceptable salts of morphine and oxycodone.

15. The method of claim 13, wherein the ratio of morphine to oxycodone is about 3 to about 2.

16. The method of claim 13, wherein the morphine and oxycodone are present in amounts of about 3 mg morphine and about 2 mg oxycodone, about 6 mg morphine and about 4 mg oxycodone, about 12 mg morphine and about 8 mg oxycodone and about 18 mg morphine and about 12 mg oxycodone.

17. The method of claim 16, wherein the composition is administered orally.

18. The method of claim 13, wherein the composition is administered orally, rectally, parenterally, sublingually, buccally, intravenously, intraarticularly, intramuscularly, intradermally, subcutaneously, intraocularly, intraperitoneally, epidurally, transdermally or by inhalation.
